(19) United States
(12) Patent Application Publication
(10) Pub. No.: US 2014/0106427 A1
(43) Pub. Date: Apr. 17, 2014

(54) Biogas From Enzyme-Treated Bagasse

(71) Applicant: Morten Gjermansen, Greve (DK)

(72) Inventor: Morten Gjermansen, Greve (DK)

(73) Assignee: Novozymes A/S, Bagsvaerd (DK)

(21) Appl. No.: 14/124,786

(22) PCT Filed: Jun. 27, 2012
(86) PCT No.: PCT/EP2012/062430

§ 371 Date: Dec. 9, 2013

(30) Foreign Application Priority Data
Jun. 28, 2011 (EP) 11171777.3

(51) Int. Cl. C12P 5/02

(52) U.S. Cl. CPC C12P 5/023
USPC 435/167

(57) ABSTRACT

The present invention relates to a process for treatment of a bagasse-derived material which treatment increases the degradability of the lignocellulosic fibres. In particular the invention relates to methane production from enzymatically treated bagasse-derived material, where the enzyme-treatment of the invention is used to increase the methane production in comparison with untreated bagasse.

### FIELD OF THE INVENTION

The present invention relates to a process for treatment of a bagasse-derived material comprising lignocellulosic fibres in which the treatment increases the degradability of the lignocellulosic fibres. In particular the invention relates to methane production from bagasse, where the enzyme-treatment of the invention is used to increase the methane production in comparison with untreated bagasse.

### BACKGROUND OF THE INVENTION

Most natural plant based material comprises a significant amount of lignocellulosic fibres that are undigestible or only slowly digestible in many biological systems. This has the consequence that for many biological processes converting plant based material a significant fraction of the treated material will not be digested or only digested in a low degree during the treatment.

Bagasse is the fibrous matter that remains after sugarcane or sorghum stalks are crushed to extract their juice. It is currently used as a biofuel and as a renewable resource in the manufacture of pulp and paper products and building materials. For each 10 tonnes of sugarcane crushed, a sugar factory produces nearly 3 tonnes of wet bagasse. Since bagasse is a by-product of the cane sugar industry, the quantity of production in each country is in line with the quantity of sugarcane produced. The moisture content of bagasse is typically 40 to 50% and the solid content is made up of about 45-50% cellulose, 20-25% hemicellulose, 18-24% lignin and 1-4% ash on a washed and dried basis.

### SUMMARY OF THE INVENTION

The invention relates to a biogas production process based on bagasse-derived material, wherein said process comprises at least one enzymatic pre-treatment of the material prior to the anaerobic biogas-producing fermentation in the digester tank, or at least one enzymatic treatment step in the biogas digester tank either prior to or during the anaerobic fermentation.

Accordingly, in a first aspect, the invention relates to a biogas production process comprising the steps of providing a slurry comprising a bagasse-derived material and water, and:
(a) performing a pre-treatment comprising adding one or more enzyme to the slurry to degrade the bagasse-derived material at a suitable temperature and pH, and then adding the enzyme-degraded material to a biogas digester tank at a suitable rate and ratio to effectively convert the material to biogas in the digester; or
(b) adding one or more enzyme to the slurry before adding the slurry to a biogas digester tank, or
(c) adding one or more enzyme to the digester tank after adding the slurry to the digester tank, to degrade the bagasse-derived material at a suitable temperature and pH and to effectively convert the material to biogas in the digester.

Several advantages are provided by the process of the invention, including, but not limited to:
Higher conversion rate in the biogas digester tank.
Higher productivity per unit of volume in the digester tank.
Lower investment in tank capacity.
Higher gas production per tank volume.
More efficient conversion of the bagasse-derived material at higher dry matter concentration.
Reduced amounts of unconverted material in the purge.
Higher dry matter content in the unconverted solids.
No need for post-converter or storage tank.
Easier dewatering of unconverted material.
Easier cleaning of the gas phase.

### DEFINITIONS

### Biogas

The term “biogas” is according to the invention intended to mean the gas obtained in a conventional anaerobic fermentor. The main component of biogas is methane and the terms “biogas” and “methane” are in this application and claims used interchangeably. The term “primary digester” is in this application and claims intended to mean the container wherein the first anaerobic fermentation takes place.

The term “secondary digester” is in this application and claims intended to mean the container wherein the second anaerobic fermentation takes place. Depending on the particular configuration of the biogas facility the primary digester may also serve as the secondary digester.

### Bagasse-Derived Material:

The term “bagasse-derived material” is in this application and claims intended to mean any material comprising bagasse or material derived therefrom, in any form, amount or ratio. The bagasse-derived material may comprise other plant derived components. The solids part of bagasse is typically made up of about 45-50% cellulose, 20-25% hemicellulose, 18-24% lignin and 1-4% ash. Typical plant derived components are starch, glucans, arabans, galactans, pectins, mannans, galactomannans and hemicelluloses such as xylans. The bagasse-derived material may be any treated or untreated material derived from bagasse as well as any composition comprising such material.

### Pre-treatment:

The term “pre-treatment” is intended to include any suitable treatment of the material prior to the actual biogas producing step. The bagasse-derived material, which may simply be bagasse from sugar-cane, may be pre-treated in any suitable way. The pre-treatment is carried out before or at the same time as the enzymatic hydrolysis. The purpose of the pre-treatment is to reduce the particle size, separate and/or release cellulose; hemicellulose and/or lignin and in this way increase the rate of hydrolysis. Pre-treatment processes such as wet-oxidation and alkaline pre-treatment targets lignin, while dilute acid and auto-hydrolysis targets hemicellulose. Steam explosion is an example of a pre-treatment that targets lignin.

The pre-treatment step may be a conventional pre-treatment step using techniques well known in the art, such as, milling or wet milling. In a preferred embodiment pre-treatment takes place in a slurry of bagasse-derived material and water. The bagasse-derived material may during pre-treatment be present in an amount between 10-80 wt. %, preferably between 20-70 wt.-%, especially between 30-60 wt.-%, such as around 50 wt-%.

Chemical, Mechanical and/or Biological Pre-treatment

The bagasse-derived material may according to the invention be chemically, mechanically and/or biologically pre-treated before hydrolysis in accordance with the process of the invention. Mechanical pre-treatment (often referred to as “physical”—pre-treatment) may be carried out alone or may be combined with other pre-treatment processes. Preferably, the chemical, mechanical and/or biological pre-treatment is carried out prior to the hydrolysis. Alternatively, the chemical, mechanical and/or biological pre-treatment may be carried out simultaneously with hydrolysis, such as simultaneously with addition of one or more hydrolyzing enzymes, and/or other enzyme activities, to release fermentable sugars, such as glucose and/or maltose.

### Chemical Pre-treatment

The term “chemical pre-treatment” refers to any chemical pre-treatment which promotes the separation and/or release of cellulose, hemicellulose and/or lignin. Examples of suitable chemical pre-treatments include treatment with; for example, dilute acid, lime, alkaline, organic solvent, ammonia, sulfur dioxide, carbon dioxide. Further, wet oxidation and pH-controlled hydrothermolysis are also considered chemical pre-treatment.

Other pre-treatment techniques are also contemplated according to the invention. Cellulose solvent treatment has been shown to convert about 90% of cellulose to glucose. It has also been shown that enzymatic hydrolysis could be greatly enhanced when the lignocellulose structure is disrupted. Alkaline H₂O₂, ozone, organosolv (uses Lewis acids, FeCl₃, Al₂(SO₄)₃ in aqueous alcohols), glycerol, dioxane, phenol, or ethylene glycol are among solvents known to disrupt cellulose structure and promote hydrolysis (Mosier et al. Bioresource Technology 96 (2005), p. 673-686).

Alkaline chemical pre-treatment with base, e.g., NaOH, Na₂CO₃, NaHCO₃, Ca(OH)₂, lime hydrate, ammonia and/or KOH or the like, is also within the scope of the invention. Pre-treatment processes using ammonia are described in, e.g., WO 2006/110891, WO 2006/11899, WO 2006/11900, WO 2006/110901, which are hereby incorporated by reference. Also the Kraft pulping process as described for example in “Pulp Processes” by Sven A. Rydholm, page 583-648. ISBN 0-89874-856-9 (1985) might be used. The solid pulp (about 50% by weight based on the dry wood chips) is collected and washed before the enzymatic treatments.

Wet oxidation techniques involve use of oxidizing agents, such as: sulphite based oxidizing agents or the like. Examples of solvent pre-treatments include treatment with DMSO (Dimethyl Sulfoxide) or the like. Chemical pre-treatment is generally carried out for 1 to 60 minutes, such as from 5 to 30 minutes, but may be carried out for shorter or longer periods of time dependent on the material to be pre-treated.

Other examples of suitable pre-treatment processes are described by Schell et al. (2003) Appl. Biochem and Biotechn. Vol. 105-108, p. 69-85, and Mosier et al. Bioresource Technology 96 (2005) 673-686, and US publication no. 2002/0164730, which references are hereby all incorporated by reference.

### Mechanical Pre-treatment

The term “mechanical pre-treatment” refers to any mechanical (or physical) pre-treatment which promotes the separation and/or release of cellulose, hemicellulose and/or lignin from bagasse-derived material. For example, mechanical pre-treatment includes various types of milling, irradiation, steaming/steam explosion, and hydrothermolysis.

Mechanical pre-treatment includes comminution (mechanical reduction of the size). Comminution includes dry milling, wet milling and vibratory ball milling. Mechanical pre-treatment may involve high pressure and/or high temperature (steam explosion). In an embodiment of the invention high pressure means pressure in the range from 300 to 600 psi, preferably 400 to 500 psi, such as around 450 psi. In an embodiment of the invention high temperature means temperatures in the range from about 100 to 300° C., preferably from about 140 to 235° C. In a preferred embodiment mechanical pre-treatment is carried out as a batch-process, in a steam gun hydrolyzer system which uses high pressure and high temperature as defined above. A Sunds Hydrolyzer (available from Sunds Defibrator AB (Sweden) may be used for this.

In a preferred embodiment the bagasse-derived material is subjected to a irradiation pre-treatment. The term “irradiation pre-treatment” refers to any pre-treatment by microwave e.g. as described by Zhu et al. “Production of ethanol from microwave-assisted alkali pre-treated wheat straw” in Process Biochemistry 41 (2006) 869-873 or ultrasonic pre-treatment, e.g., as described by e.g. Li et al. “A kinetic study on enzymatic hydrolysis of a variety of pulps for its enhancement with continuous ultrasonic irradiation”, in Biochemical Engineering Journal 19 (2004) 155-164. Preferably, the bagasse-derived material prior to step (a) or (b) has been subjected to a microwave and/or an ultrasonic irradiation treatment.

In another preferred embodiment, the bagasse-derived material or the slurry is homogenized; preferably by milling, wet-milling, grinding or wet-grinding prior to or during step (a) or prior to step (b).

### Combined Chemical and Mechanical Pre-treatment

In a preferred embodiment the bagasse-derived material is subjected to both chemical and mechanical pre-treatment. For instance, the pre-treatment step may involve dilute or mild acid treatment and high temperature and/or pressure treatment. The chemical and mechanical pre-treatments may be carried out sequentially or simultaneously, as desired.

In a preferred embodiment the pre-treatment is carried out as a dilute and/or mild acid steam explosion step. In another preferred embodiment pre-treatment is carried out as an ammonia fiber explosion step (or AFEX pre-treatment step).

In yet another preferred embodiment, a base is added to the bagasse-derived material or the slurry prior to or while it is being homogenized; preferably the base is NaOH, Na₂CO₃, NaHCO₃, Ca(OH)₂, lime hydrate, ammonia and/or KOH.

### Biological Pre-Treatment

The term “biological pre-treatment” refers to any biological pre-treatment which promotes the separation and/or release of cellulose, hemicellulose, and/or lignin from the bagasse-derived material. Known biological pre-treatment techniques involve applying lignin-solubilizing microorganisms (see, for example, Hsu, T.-A., 1996, Pretreatment of biomass, in Handbook on Bioethanol: Production and Utilization, Wyman, C. E., ed., Taylor & Francis, Washington, D.C., 179-212; Ghosh, P., and Singh, A., 1993, Physicochemical and biological treatments for enzymatic/microbial conversion of lignocellulosic biomass, Adv. Appl. Microbiol. 39: 295-333; McMillan, J. D., 1994, Pretreating lignocellulosic biomass: a review, in Enzymatic Conversion of Biomass for Fuels Production, Himmel, M. E., Baker, J. O., and Overend, R. P., eds., ACS Symposium Series 566, American Chemical Society, Washington, D.C., chapter 15; Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson, L., and Hahn-Hagerdal, B., 1996, Fermentation of lignocellulosic hydrolysates for ethanol production, Enz. Microb. Tech. 18: 312-331; and Vallander, L., and Eriksson, K.-E. L., 1990, Production of ethanol from lignocellulosic materials: State of the Art Adv. Biochem. Eng./Biotechnol. 42: 63-95).

In a preferred embodiment, the bagasse-derived material has been chemically, mechanical and/or biologically treated prior to step (a) or (b).

### DETAILED DESCRIPTION OF THE INVENTION

In a first aspect, the invention relates to a biogas production process comprising the steps of providing a slurry comprising a bagasse-derived material and water, and:
(a) performing a pre-treatment comprising adding one or more enzyme to the slurry to degrade the bagasse-derived material at a suitable temperature and pH, and then adding the enzyme-degraded material to a biogas digester tank at a suitable rate and ratio to effectively convert the material to biogas in the digester; or
(b) adding one or more enzyme to the slurry before adding the slurry to a biogas digester tank, or adding one or more enzyme to the digester tank after adding the slurry to the digester tank, to degrade the bagasse-derived material at a suitable temperature and pH and to effectively convert the material to biogas in the digester.

### Enzymatic Hydrolysis

Before or while bagasse-derived material is fermented it is hydrolyzed enzymatically to break down especially hemicellulose and/or cellulose into fermentable sugars. During enzymatic liquefaction polysaccharides like starch, hemicelluloses, mannan and cellulose are solubilised and converted mainly to oligosaccharides, any protein is hydrolysed mainly to peptides and cellulose is converted to cellodextrins. This may be achieved in an enzymatic pre-treatment.

Before or during the pre-treatment, a milling of the biomass may be done, preferably a wet grinding, optionally facilitated by addition of the enzymes according to the invention. Temperature and pH is adjusted to allow the enzymes to function. The bagasse-derived material to be hydrolyzed typically constitutes above 2.5% wt-% DS (dry solids), preferably above 5% wt-% DS, preferably above 10% wt-% DS, preferably above 15 wt-% DS, preferably above 20 wt.-% DS, more preferably above 25 wt-% DS of a slurry.

Preferably, the content of bagasse-derived material in the slurry is adjusted by continuous or stepwise addition of material to the slurry during step (a) or (b).

In a preferred embodiment, a solids separation step is performed in step (a) after the bagasse-derived material is degraded but before it is added to the digester tank, to purge not-solubilized solids and optionally feed them back into step (a) of the process.

The bagasse-derived material may further be subjected to the action of one, or several or all enzyme activities selected from the group consisting of an amylolytic enzyme, a lipolytic enzyme, a proteolytic enzyme, a cellulolytic enzyme, an oxidoreductase and a plant cell-wall degrading enzyme.

In a preferred embodiment, the one or more enzyme is selected from the group consisting of aminopeptidase, alpha-amylase, amyloglucosidase, arabinofuranosidase, arabinoxylanase, beta-glucanase, carbohydrase, carboxypeptidase, catalase, cellobiohydrolase, cellulase, chitinase, cutinase, cyclodextrin glycosyltransferase, ferulic acid esterase, deoxyribonuclease, endo-cellulase, endo-glucanase, endo-xylanase, esterase, galactosidase, beta-galactosidase, glucoamylase, glucose oxidase, glucosidase, haloperoxidase, hemicellulase, invertase, isomerase, laccase, ligase, lipase, lyase, mannanase, mannosidase, oxidase, pectate lyase, pectin lyase, pectin trans-eliminase, pectin ethylesterase, pectin methylesterase, pectinolytic enzyme, peroxidase, protease, phytase, phenoloxidase, polygalacturonase, polyphenoloxidase, proteolytic enzyme, rhamnogalacturonan lyase, rhamnoglucanase, rhamnogalacturonase, ribonuclease, SPS-ase, transferase, transglutaminase, xylanase and xyloglucanase.

In another preferred embodiment, the one or more enzyme is a protease, a pectate lyase, a ferulic acid esterase and/or a mannanase.

From a pre-treatment tank, enzymatically liquefied material is fed to a biogas digester tank in a rate and ratio that fits with the conversion rate to gas. In the liquefaction system, pH is kept at same pH as in the digester tank.

It is noteworthy, that the pre-treated biomass material should preferably have a neutral to basic pH value when it is added to the biogas digester, it is thought that addition of acidic biomass may halt the biogas conversion process due to inhibition of the common methanogenic microorganisms.

In a preferred embodiment of the method of the first aspect, the pH is between 7 and 10, such as from 7.6 to 10; preferably from 8 to 10, or from 8 to 9, preferably around pH 8.5. The pH may be adjusted using NaOH, Na₂CO₃, NaHCO₃, Ca(OH)₂, lime hydrate, ammonia and/or KOH. The temperature may be between 20-70° C., preferably 30-60° C., and more preferably 40-55° C., e.g., around 50° C. In a hydrolysis step, cell walls are degraded and the cellulose fibrils are made accessible for further hydrolysis. The hydrolysis step may be carried out as a fed batch process where pre-treated bagasse-derived material is fed continuously/gradually or stepwise into a solution containing hydrolyzing enzymes.

In an embodiment a pectate lyase, a ferulic acid esterase, and a mannanase is present in a second hydrolysis step in the pre-treatment. In an embodiment a pectate lyase, a ferulic acid esterase, mannanase and a cellulase is present. In an embodiment a pectate lyase, a ferulic acid esterase, mannanase, a cellulase and a protease is present.

Optionally, cellulose fibrils may be isolated and treated with an alkaline endo-glucanase composition under neutral to basic pH conditions. In that step, the dry solids (DS) is preferably above 10 wt.-% DS, preferably above 15 wt-% DS, preferably above 20 wt.-% DS, more preferably above 25 wt-% DS.

The pH should be between 7 and 10, such as from 8 to 9, preferably around pH 8.5. Prior to steps (a) or (b) the pH may be adjusted using NaOH, Na₂CO₃, NaHCO₃, Ca(OH)₂, lime hydrate, ammonia and/or KOH. The temperature may be between in range from 20-70° C., preferably 30-60° C., and more preferably 40-50° C.

The cellulose fibrils may be treated with a cellulase composition comprising cellulolytic activity under neutral to acid pH conditions. Preferably the pH is between 4-7, preferably 5-7, such as around 5.5. The pH is preferably adjusted using phosphoric acid, succinic acid, hydrochloric acid and/or sulphuric acid. Preferably with a temperature in the range of 20-70° C., preferably 30-60° C., and more preferably 40-50° C.

### Enzymes

Even if not specifically mentioned in context of a process or process of the invention, it is to be understood that the enzyme(s) as well as other compounds are used in an “effective amount”.

### Proteases

Any protease suitable for use under alkaline conditions can be used. Suitable proteases include those of animal, vegetable or microbial origin. Microbial origin is preferred. Chemically or genetically modified mutants are included. The protease may be a serine protease, preferably an alkaline microbial protease or a trypsin-like protease. Examples of alkaline proteases are subtilisins, especially those derived from Bacillus, e.g., subtilisin Novo, subtilisin Carlsberg, subtilisin 309, subtilisin 147 and subtilisin 168 (described in WO 89/06279). Examples of trypsin-like proteases are trypsin (e.g. of porcine or bovine origin) and the Fusarium protease described in WO 89/06270.

Preferred commercially available protease enzymes include those sold under the trade names Everlase™, Kannase™, Alcalase™, Savinase™, Primase™, Durazym™, and Esperase™ by Novozymes NS (Denmark), those sold under the tradename Maxatase, Maxacal, Maxapem, Properase, Purafect and Purafect OXP by Genencor International, and those sold under the tradename Opticlean and Optimase by Solvay Enzymes.

### Hemicellulolytic Enzymes

Any hemicellulase suitable for use in hydrolyzing hemicellulose, may be used. Preferred hemicellulases include pectate lyases, xylanases, arabinofuranosidases, acetyl xylan esterase, ferulic acid esterase, glucuronidases, endo-galactanase, mannases, endo or exo arabinases, exo-galactanses, and mixtures of two or more thereof. Preferably, the hemicellulase for use in the present invention is an endo-acting hemicellulase, and more preferably, the hemicellulase is an endo-acting hemicellulase which has the ability to hydrolyze hemicellulose under basic conditions of above pH 7, preferably pH 7-10.

In an embodiment the hemicellulase is a xylanase. In an embodiment the xylanase may preferably be of microbial origin, such as of fungal origin (e.g., Trichoderma, Meripilus, Humicola, Aspergillus, Fusarium) or from a bacterium (e.g., Bacillus). In a preferred embodiment the xylanase is derived from a filamentous fungus, preferably derived from a strain of Aspergillus, such as Aspergillus aculeatus; or a strain of Humicola, preferably Humicola lanuginosa. The xylanase may preferably be an endo-1,4-beta-xylanase, more preferably an endo-1,4-beta-xylanase of GH10 or GH11. Examples of commercial xylanases include SHEARZYME® 200 L, SHEARZYME® 500 L, BIOFEED WHEAT®, and PULPZYME™ HC (from Novozymes) and GC 880, SPEZYME® CP (from Genencor Int).

The hemicellulase may be added in an amount effective to hydrolyze hemicellulose, such as, in amounts from about 0.001 to 0.5 wt.-% of total solids (TS), more preferably from about 0.05 to 0.5 wt.-% of TS.

Xylanases may be added in the amounts of 1.0-1000 FXU/kg dry solids, preferably from 5-500 FXU/kg dry solids, preferably from 5-100 FXU/kg dry solids and most preferably from 10-100 FXU/kg dry solids.

Xylanases may alternatively be added in amounts of 0.001-1.0 g/kg DS substrate, preferably in the amounts of 0.005-0.5 g/kg DS substrate, and most preferably from 0.05-0.10 g/kg DS substrate.

### Pectolytic Enzymes (or Pectinases)

Any pectinolytic enzyme that can degrade the pectin composition of plant cell walls may be used in practicing the present invention. Suitable pectinases include, without limitation, those of fungal or bacterial origin. Chemically or genetically modified pectinases are also encompassed. Preferably, the pectinase used in the invention are recombinantly produced and are mono-component enzymes.

Pectinases can be classified according to their preferential substrate, highly methyl-esterified pectin or low methyl-esterified pectin and polygalacturonic acid (pectate), and their reaction mechanism, beta-elimination or hydrolysis. Pectinases can be mainly endo-acting, cutting the polymer at random sites within the chain to give a mixture of oligomers, or they may be exo-acting, attacking from one end of the polymer and producing monomers or dimers. Several pectinase activities acting on the smooth regions of pectin are included in the classification of enzymes provided by Enzyme Nomenclature (1992), e.g., pectate lyase (EC 4.2.2.2), pectin lyase (EC 4.2.2.10), polygalacturonase (EC 3.2.1.15), exo-polygalacturonase (EC 3.2.1.67), exo-polygalacturonate lyase (EC 4.2.2.9) and exo-poly-alpha-galacturonosidase (EC 3.2.1.82).

In embodiments the pectinase is a pectate lyase. Pectate lyase enzymatic activity as used herein refers to catalysis of the random cleavage of alpha-1,4-glycosidic linkages in pectic acid (also called polygalcturonic acid) by transelimination. Pectate lyases are also termed polygalacturonate lyases and poly(1,4-α-D-galacturonide) lyases.

The Pectate lyase (EC 4.2.2.2) is an enzyme which catalyse the random cleavage of α-1,4-glycosidic linkages in pectic acid (also called polygalacturonic acid) by transelimination. Pectate lyases also include polygalacturonate lyases and poly(1,4-α-D-galacturonide) lyases. Examples of preferred pectate lyases are those that have been cloned from different bacterial genera such as Erwinia, Pseudomonas, Klebsiella, Xanthomonas and Bacillus, especially Bacillus licheniformis (U.S. Pat. No. 6,124,127), as well as from Bacillus subtilis (Nasser et al. (1993) FEBS Letts. 335:319-326) and Bacillus sp. YA-14 (Kim et al. (1994) Biosci. Biotech. Biochem. 58:947-949). Purification of pectate lyases with maximum activity in the pH range of 8-10 produced by Bacillus pumilus (Dave and Vaughn (1971) J. Bacteriol. 108:166-174), B. polymyxa (Nagel and Vaughn (1961) Arch. Biochem. Biophys. 93:344-352), B. stearothermophilus (Karbassi and Vaughn (1980) Can. J. Microbiol. 26:377-384), Bacillus sp. (Hasegawa and Nagel (1966) J. Food Sci. 31:838-845) and Bacillus sp. RK9 (Kelly and Fogarty (1978) Can. J. Microbiol. 24:1164-1172) have also been described.

A preferred pectate lyase may be obtained from Bacillus licheniformis as described in U.S. Pat. No. 6,124,127.

Other pectate lyases could be those that comprise the amino acid sequence of a pectate lyase disclosed in Heffron et al., (1995) Mol. Plant-Microbe Interact. 8: 331-334 and Henrissat et al., (1995) Plant Physiol. 107: 963-976.

A single enzyme or a combination of pectate lyases may be used. A preferred commercial pectate lyase preparation suitable for the invention is BioPrep® 3000 L available from Novozymes NS.

### Mannanases

In the context of the present invention a mannanase is a beta-mannanase and defined as an enzyme belonging to EC 3.2.1.78.

Mannanases have been identified in several Bacillus organisms. For example, Talbot et al., Appl. Environ. Microbiol., Vol. 56, No. 11, pp. 3505-3510 (1990) describes a beta-mannanase derived from Bacillus stearothermophilus having an optimum pH of 5.5-7.5. Mendoza et al., World J. Microbiol. Biotech., Vol. 10, No. 5, pp. 551-555 (1994) describes a beta-mannanase derived from Bacillus subtilis having an optimum activity at pH 5.0 and 55° C. JP-03047076 discloses a beta-mannanase derived from Bacillus sp., having an optimum pH of 8-10. JP-63056289 describes the production of an alkaline, thermostable beta-mannanase. JP-08051975 discloses alkaline beta-mannanases from alkalophilic Bacillus sp. AM-001. A purified mannanase from Bacillus amyloliquefaciens is disclosed in WO 97/11164. WO 94/25576 discloses an enzyme from Aspergillus aculeatus, CBS 101.43, exhibiting mannanase activity and WO 93/24622 discloses a mannanase isolated from Trichoderma reesei.

The mannanase may be derived from a strain of the genus Bacillus, such as the amino acid sequence having the sequence deposited as GENESEQP accession number AAY54122 or an amino acid sequence which is homologous to this amino acid sequence. A suitable commercial mannanase preparation is Mannaway® produced by Novozymes A/S.

### Ferulic Esterases

In the context of the present invention a ferulic esterase is defined as an enzyme belonging to EC 3.1.1.73.

A suitable ferulic esterase preparation can be obtained from Malabrancea, e.g., from P. cinnamomea, such as e.g. a preparation comprising the ferulic esterase having the amino acid sequence shown in SEQ ID NO:2 in European patent application number 07121322.7, or an amino acid sequence which is homologous to this amino acid sequence.

Another suitable ferulic esterase preparation can be obtained from Penicillium, e.g., from P. aurantiogriseum, such as e.g. a preparation comprising the ferulic esterase having the amino acid sequence shown in SEQ ID NO:2 in European patent application number 0815469.7, or an amino acid sequence which is homologous to this amino acid sequence. A suitable commercial ferulic esterase preparation preparation is NOVOZYM® 342 L produced by Novozymes A/S.

### Alkaline Endo-Qlucanases

The term “endoglucanase” means an endo-1,4-(1,3;1,4)-beta-D-glucan 4-glucanohydrolase (E.C. 3.2.1.4), which catalyses endo-hydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3 glucans such as cereal beta-D-glucans or xyloglucans, and other plant material containing cellulosic components. Alkaline endo-glucanases are endo-glucanases having activity under alkaline conditions.

In a preferred embodiment endoglucanases may be derived from a strain of the genus Trichoderma, preferably a strain of Trichoderma reesei; a strain of the genus Humicola, such as a strain of Humicola insolens; or a strain of Chrysosporium, preferably a strain of Chrysosporium lucknowense.

In a preferred embodiment endoglucanases may be derived from a strain of the genus Bacillus akibai.

In an embodiment the alkaline endo-glucanase composition is one of the commercially available products CAREZYME®, ENDOLASE® and CELLUCLEAN® (Novozymes A/S, Denmark). The enzyme may be applied in a dosage of 1-100 g/kg cellulose.

### Acid Cellulolytic Activity

The term “acid cellulolytic activity” as used herein are understood as comprising enzymes having cellobiohydrolase activity (EC 3.2.1.91), e.g., cellobiohydrolase I and/or cellobiohydrolase II, as well as endo-glucanase activity (EC 3.2.1.4) and/or beta-glucosidase activity (EC 3.2.1.21) having activity at pH below 6.

The cellulolytic activity may, in a preferred embodiment, be in the form of a preparation of enzymes of fungal origin, such as from a strain of the genus Trichoderma, preferably a strain of Trichoderma reesei; a strain of the genus Humicola, such as a strain of Humicola insolens; or a strain of Chrysosporium, preferably a strain of Chrysosporium lucknowense.

In preferred embodiment the cellulolytic enzyme preparation contains one or more of the following activities: endoglucanase, cellobiohydrolases I and II, and beta-glucosidase activity.

In a preferred embodiment cellulolytic enzyme preparation is a composition disclosed in WO2008/151079, which is hereby incorporated by reference. In a preferred embodiment the cellulolytic enzyme preparation comprising a polypeptide having cellulolytic enhancing activity, preferably a family GH61A polypeptide, preferably those disclosed in WO 2005/074656 (Novozymes). The cellulolytic enzyme preparation may further comprise beta-glucosidase, such as beta-glucosidase derived from a strain of the genus Trichoderma, Aspergillus or Penicillium, including the fusion protein having beta-glucosidase activity disclosed in WO 2008/057637 (Novozymes). In a preferred embodiment the cellulolytic enzyme preparation may also comprises a CBH II enzyme, preferably Thielavia terrestris cellobiohydrolase II (CEL6A). In another preferred embodiment the cellulolytic enzyme preparation may also comprise cellulolytic enzymes; preferably those derived from Trichoderma reesei or Humicola insolens.

The cellulolytic enzyme composition may also comprise a polypeptide having cellulolytic enhancing activity (GH61A) disclosed in WO 2005/074656; an Aspergillus oryzae beta-glucosidase fusion protein (WO 2008/057637), and cellulolytic enzymes derived from Trichoderma reesei.

The cellulolytic enzyme composition may also comprise an Aspergillus aculeatus GH10 xylanase (WO 94/021785) and a Trichoderma reesei cellulase preparation containing Aspergillus fumigatus beta-glucosidase (WO 2005/047499) and Thermoascus aurantiacus GH61A polypeptide (WO 2005/074656).

The cellulolytic enzyme composition may also comprise an Aspergillus aculeatus GH10 xylanase (WO 94/021785) and/or a Trichoderma reesei cellulase preparation.

In another preferred embodiment the cellulolytic composition comprising a polypeptide having cellulolytic enhancing activity (GH61A) disclosed in WO 2005/074656; an Aspergillus oryzae beta-glucosidase fusion protein (WO 2008/057637), Thielavia terrestris cellobiohydrolase II (CEL6A), and cellulolytic enzymes preparation derived from Trichoderma reesei.

In an embodiment the cellulolytic enzyme composition is the commercially available product CELLUCLAST™ 1.5 L, CELLUZYME™, Cellic™ CTec, Cellic™ CTec2, Cellic™ HTec, Cellic™ HTec2 (all Novozymes A/S, Denmark) or ACCELLARASE™ 1000 (Genencor Int, Inc., USA).

The cellulolytic activity may be dosed in the range from 0.1-100 FPU per gram total solids (TS), preferably 0.5-50 FPU per gram TS, especially 1-20 FPU per gram TS.

### Cellulase Activity Using Filter Paper Assay (FPU Assay)

The process is disclosed in a document entitled “Measurement of Cellulase Activities” by Adney, B. and Baker, J. 1996. Laboratory Analytical Procedure, LAP-006, National Renewable Energy Laboratory (NREL). It is based on the IUPAC process for measuring cellulase activity (Ghose, T. K., Measurement of Cellulse Activities, Pure & Appl. Chem. 59, pp. 257-268, 1987.

The process is carried out as described by Adney and Baker, 1996, supra, except for the use of a 96 well plates to read the absorbance values after color development, as described below:

### Enzyme Assay Tubes:

A rolled filter paper strip (#1 Whatman; 1×6 cm; 50 mg) is added to the bottom of a test tube (13×100 mm).
To the tube is added 1.0 mL of 0.05 M Na-citrate buffer (pH 4.80).
The tubes containing filter paper and buffer are incubated 5 min. at 50° C. (±0.1° C.) in a circulating water bath.
Following incubation, 0.5 mL of enzyme dilution in citrate buffer is added to the tube. Enzyme dilutions are designed to produce values slightly above and below the target value of 2.0 mg glucose.
The tube contents are mixed by gently vortexing for 3 seconds.
After vortexing, the tubes are incubated for 60 mins. at 50° C. (±0.1° C.) in a circulating water bath.
Immediately following the 60 min. incubation, the tubes are removed from the water bath, and 3.0 mL of DNS reagent is added to each tube to stop the reaction. The tubes are vortexed 3 seconds to mix.

### Blank and Controls:

A reagent blank is prepared by adding 1.5 mL of citrate buffer to a test tube.
A substrate control is prepared by placing a rolled filter paper strip into the bottom of a test tube, and adding 1.5 mL of citrate buffer.
Enzyme controls are prepared for each enzyme dilution by mixing 1.0 mL of citrate buffer with 0.5 mL of the appropriate enzyme dilution.
The reagent blank, substrate control, and enzyme controls are assayed in the same manner as the enzyme assay tubes, and done along with them.

### Glucose Standards:

A 100 mL stock solution of glucose (10.0 mg/mL) is prepared, and 5 mL aliquots are frozen. Prior to use, aliquots are thawed and vortexed to mix.
Dilutions of the stock solution are made in citrate buffer as follows:

G1=1.0 mL stock+0.5 mL buffer=6.7 mg/mL=3.3 mg/0.5 mL

G2=0.75 mL stock+0.75 mL buffer=5.0 mg/mL=2.5 mg/0.5 mL

G3=0.5 mL stock+1.0 mL buffer=3.3 mg/mL=1.7 mg/0.5 mL

G4=0.2 mL stock+0.8 mL buffer=2.0 mg/mL=1.0 mg/0.5 mL
Glucose standard tubes are prepared by adding 0.5 mL of each dilution to 1.0 mL of citrate buffer.
The glucose standard tubes are assayed in the same manner as the enzyme assay tubes, and done along with them.

### Color Development:

Following the 60 min. incubation and addition of DNS, the tubes are all boiled together for 5 mins. in a water bath.
After boiling, they are immediately cooled in an ice/water bath.
When cool, the tubes are briefly vortexed, and the pulp is allowed to settle. Then each tube is diluted by adding 50 microL from the tube to 200 microL of ddH2O in a 96-well plate. Each well is mixed, and the absorbance is read at 540 nm.
   Calculations (examples are given in the NREL document)
A glucose standard curve is prepared by graphing glucose concentration (mg/0.5 mL) for the four standards (G1-G4) vs. A540. This is fitted using a linear regression (Prism Software), and the equation for the line is used to determine the glucose produced for each of the enzyme assay tubes.
A plot of glucose produced (mg/0.5 mL) vs. total enzyme dilution is prepared, with the Y-axis (enzyme dilution) being on a log scale.
A line is drawn between the enzyme dilution that produced just above 2.0 mg glucose and the dilution that produced just below that. From this line, it is determined the enzyme dilution that would have produced exactly 2.0 mg of glucose.
The Filter Paper Units/mL (FPU/mL) are calculated as follows:

FPU/mL=0.37/enzyme dilution producing 2.0 mg glucose

### Cellulolytic Enhancing Activity

The term “cellulolytic enhancing activity” is defined herein as a biological activity that enhances the hydrolysis of a bagasse-derived material by proteins having cellulolytic activity. For purposes of the present invention, cellulolytic enhancing activity is determined by measuring the increase in reducing sugars or in the increase of the total of cellobiose and glucose from the hydrolysis of a bagasse-derived material, e.g., pre-treated bagasse-derived material by cellulolytic protein under the following conditions: 1-50 mg of total protein/g of cellulose in PCS (pre-treated corn stover), wherein total protein is comprised of 80-99.5% w/w cellulolytic protein/g of cellulose in PCS and 0.5-20% w/w protein of cellulolytic enhancing activity for 1-7 day at 50° C. compared to a control hydrolysis with equal total protein loading without cellulolytic enhancing activity (1-50 mg of cellulolytic protein/g of cellulose in PCS).

The polypeptides having cellulolytic enhancing activity enhance the hydrolysis of a bagasse-derived material catalyzed by proteins having cellulolytic activity by reducing the amount of cellulolytic enzyme required to reach the same degree of hydrolysis preferably at least 0.1-fold, more at least 0.2-fold, more preferably at least 0.3-fold, more preferably at least 0.4-fold, more preferably at least 0.5-fold, more preferably at least 1-fold, more preferably at least 3-fold, more preferably at least 4-fold, more preferably at least 5-fold, more preferably at least 10-fold, more preferably at least 20-fold, even more preferably at least 30-fold, most preferably at least 50-fold, and even most preferably at least 100-fold.

In a preferred embodiment the hydrolysis and/or fermentation is carried out in the presence of a cellulolytic enzyme in combination with a polypeptide having cellulolytic enhancing activity. In a preferred embodiment the polypeptide having cellulolytic enhancing activity is a family GH61A polypeptide. WO 2005/074647 discloses isolated polypeptides having cellulolytic enhancing activity and polynucleotides thereof from Thielavia terrestris. WO 2005/074656 discloses an isolated polypeptide having cellulolytic enhancing activity and a polynucleotide thereof from Thermoascus aurantiacus. U.S. Published Application Serial No. 2007/0077630 discloses an isolated polypeptide having cellulolytic enhancing activity and a polynucleotide thereof from Trichoderma reesei.

### Alpha-Amylase

According to the invention any alpha-amylase may be used, such as of fungal, bacterial or plant origin. In a preferred embodiment the alpha-amylase is an acid alpha-amylase, e.g., acid fungal alpha-amylase or acid bacterial alpha-amylase. The term “acid alpha-amylase” means an alpha-amylase (E.C. 3.2.1.1) which added in an effective amount has activity optimum at a pH in the range of 3 to 7, preferably from 3.5 to 6, or more preferably from 4-5.

### Bacterial Alpha-Amylase

According to the invention a bacterial alpha-amylase is preferably derived from the genus Bacillus.

In a preferred embodiment the Bacillus alpha-amylase is derived from a strain of Bacillus licheniformis, Bacillus amyloliquefaciens, Bacillus subtilis or Bacillus stearothermophilus, but may also be derived from other Bacillus sp. Specific examples of contemplated alpha-amylases include the Bacillus licheniformis alpha-amylase shown in SEQ ID NO: 4 in WO 99/19467, the Bacillus amyloliquefaciens alpha-amylase SEQ ID NO: 5 in WO 99/19467 and the Bacillus stearothermophilus alpha-amylase shown in SEQ ID NO: 3 in WO 99/19467 (all sequences hereby incorporated by reference). In an embodiment the alpha-amylase may be an enzyme having a degree of identity of at least 60%, preferably at least 70%, more preferred at least 80%, even more preferred at least 90%, such as at least 95%, at least 96%, at least 97%, at least 98% or at least 99% to any of the sequences shown in SEQ ID NOS: 1, 2 or 3, respectively, in WO 99/19467.

The Bacillus alpha-amylase may also be a variant and/or hybrid, especially one described in any of WO 96/23873, WO 96/23874, WO 97/41213, WO 99/19467, WO 00/60059, and WO 02/10355 (all documents hereby incorporated by reference). Specifically contemplated alpha-amylase variants are disclosed in U.S. Pat. Nos. 6,093,562, 6,297,038 or U.S. Pat. No. 6,187,576 (hereby incorporated by reference) and include Bacillus stearothermophilus alpha-amylase (BSG alpha-amylase) variants having a deletion of one or two amino acid in positions R179 to G182, preferably a double deletion disclosed in WO 1996/023873—see e.g., page 20, lines 1-10 (hereby incorporated by reference), preferably corresponding to delta(181-182) compared to the wild-type BSG alpha-amylase amino acid sequence set forth in SEQ ID NO:3 disclosed in WO 99/19467 or deletion of amino acids R179 and G180 using SEQ ID NO:3 in WO 99/19467 for numbering (which reference is hereby incorporated by reference). Even more preferred are Bacillus alpha-amylases, especially Bacillus stearothermophilus alpha-amylase, which have a double deletion corresponding to delta(181-182) and further comprise a N193F substitution (also denoted I181*+G182*+N193F) compared to the wild-type BSG alpha-amylase amino acid sequence set forth in SEQ ID NO:3 disclosed in WO 99/19467. In an embodiment the bacterial alpha-amylase is dosed in an amount of 0.0005-5 KNU per g DS, preferably 0.001-1 KNU per g DS, such as around 0.050 KNU per g DS.

### Fungal Alpha-Amylase

Fungal alpha-amylases include alpha-amylases derived from a strain of the genus Aspergillus, such as, Aspergillus oryzae, Aspergillus niger and Aspergillis kawachii alpha-amylases.

A preferred acidic fungal alpha-amylase is a Fungamyl-like alpha-amylase which is derived from a strain of Aspergillus oryzae. According to the present invention, the term “Fungamyl-like alpha-amylase” indicates an alpha-amylase which exhibits a high identity, i.e. at least 70%, at least 75%, at least 80%, at least 85%, at least 90%, at least 95%, at least 96%, at least 97%, at least 98%, at least 99% or even 100% identity to the mature part of the amino acid sequence shown in SEQ ID NO: 10 in WO 96/23874.

Another preferred acid alpha-amylase is derived from a strain Aspergillus niger. In a preferred embodiment the acid fungal alpha-amylase is the one from Aspergillus niger disclosed as “AMYA_ASPNG” in the Swiss-prot/TeEMBL database under the primary accession no. P56271 and described in WO 89/01969 (Example 3—incorporated by reference). A commercially available acid fungal alpha-amylase derived from Aspergillus niger is SP288 (available from Novozymes A/S, Denmark).

Other contemplated wild-type alpha-amylases include those derived from a strain of the genera Rhizomucor and Meripilus, preferably a strain of Rhizomucor pusillus (WO 2004/055178 incorporated by reference) or Meripilus giganteus.

In a preferred embodiment the alpha-amylase is derived from Aspergillus kawachii and disclosed by Kaneko et al. J. Ferment. Bioeng. 81:292-298 (1996) “Molecular-cloning and determination of the nucleotide-sequence of a gene encoding an acid-stable alpha-amylase from Aspergillus kawachii.”; and further as EMBL:#AB008370.

The fungal alpha-amylase may also be a wild-type enzyme comprising a starch-binding domain (SBD) and an alpha-amylase catalytic domain (i.e., none-hybrid), or a variant thereof. In an embodiment the wild-type alpha-amylase is derived from a strain of Aspergillus kawachii.

An acid alpha-amylases may according to the invention be added in an amount of 0.001 to 10 AFAU/g DS, preferably from 0.01 to 5 AFAU/g DS, especially 0.3 to 2 AFAU/g DS or 0.001 to 1 FAU-F/g DS, preferably 0.01 to 1 FAU-F/g DS.

### Commercial Alpha-Amylase Products

Preferred commercial compositions comprising alpha-amylase include MYCOLASE™ from DSM (Gist Brocades), BAN™, TERMAMYL™ SC, FUNGAMYL™, LIQUOZYME™ X, LIQUOZYME™ SC and SAN™ SUPER, SAN™ EXTRA L (Novozymes A/S) and CLARASE™ L-40,000, DEX-LO™, SPEZYME™ FRED, SPEZYME™ AA, and SPEZYME™ DELTA AA (Genencor Int.), and the acid fungal alpha-amylase sold under the trade name SP288 (available from Novozymes A/S, Denmark).

### Carbohydrate-Source Generating Enzyme

The term “carbohydrate-source generating enzyme” includes glucoamylase (being glucose generators), beta-amylase and maltogenic amylase (being maltose generators) and also pullulanase and alpha-glucosidase. A carbohydrate-source generating enzyme is capable of producing a carbohydrate that can be used as an energy-source by the fermenting organism(s) in question, for instance, when used in a process of the invention for producing a fermentation product, such as ethanol. The generated carbohydrate may be converted directly or indirectly to the desired fermentation product, preferably ethanol. According to the invention a mixture of carbohydrate-source generating enzymes may be used. Especially contemplated mixtures are mixtures of at least a glucoamylase and an alpha-amylase, especially an acid amylase, even more preferred an acid fungal alpha-amylase. The ratio between acid fungal alpha-amylase activity (FAU-F) and glucoamylase activity (AGU) (i.e., FAU-F per AGU) may in an embodiment of the invention be between 0.1 and 100, in particular between 2 and 50, such as in the range from 10-40.

### Glucoamylase

A glucoamylase used according to the invention may be derived from any suitable source, e.g., derived from a microorganism or a plant. Preferred glucoamylases are of fungal or bacterial origin, selected from the group consisting of Aspergillus glucoamylases, in particular Aspergillus niger G1 or G2 glucoamylase (Boel et al. (1984), EMBO J. 3 (5), p. 1097-1102), or variants thereof, such as those disclosed in WO 92/00381, WO 00/04136 and WO 01/04273 (from Novozymes, Denmark); the A. awamori glucoamylase disclosed in WO 84/02921, Aspergillus oryzae glucoamylase (Agric. Biol. Chem. (1991), 55 (4), p. 941-949), or variants or fragments thereof. Other Aspergillus glucoamylase variants include variants with enhanced thermal stability: G137A and G139A (Chen et al. (1996), Prot. Eng. 9, 499-505); D257E and D293E/Q (Chen et al. (1995), Prot. Eng. 8, 575-582); N182 (Chen et al. (1994), Biochem. J. 301, 275-281); disulphide bonds, A246C (Fierobe et al. (1996), Biochemistry, 35, 8698-8704; and introduction of Pro residues in position A435 and S436 (Li et al. (1997), Protein Eng. 10, 1199-1204.

Other glucoamylases include Athelia rolfsii (previously denoted Corticium rolfsii) glucoamylase (see U.S. Pat. No. 4,727,026 and (Nagasaka, Y. et al. (1998) “Purification and properties of the raw-starch-degrading glucoamylases from Corticium rolfsii, Appl Microbiol Biotechnol 50:323-330), Talaromyces glucoamylases, in particular derived from Talaromyces emersonii (WO 99/28448), Talaromyces leycettanus (U.S. Pat. No. Re. 32,153), Talaromyces duponti, Talaromyces thermophilus (U.S. Pat. No. 4,587,215).

Bacterial glucoamylases contemplated include glucoamylases from the genus Clostridium, in particular C. thermoamylolyticum (EP 135,138), and C. thermohydrosulfuricum (WO 86/01831) and Trametes cingulata, Pachykytospora papyracea; and Leucopaxillus giganteus all disclosed in WO 2006/069289; or Peniophora rufomarginata disclosed in PCT/US2007/066618; or a mixture thereof. Also hybrid glucoamylase are contemplated according to the invention. Examples the hybrid glucoamylases disclosed in WO 2005/045018. Specific examples include the hybrid glucoamylase disclosed in Table 1 and 4 of Example 1 (which hybrids are hereby incorporated by reference).

Contemplated are also glucoamylases which exhibit a high identity to any of above mention glucoamylases, i.e., at least 70%, at least 75%, at least 80%, at least 85%, at least 90%, at least 95%, at least 96%, at least 97%, at least 98%, at least 99% or even 100% identity to the mature enzymes sequences mentioned above.

Commercially available compositions comprising glucoamylase include AMG 200 L; AMG 300 L; SAN™ SUPER, SAN™ EXTRA L, SPIRIZYME™ PLUS, SPIRIZYME™ FUEL, SPIRIZYME™ B4U and AMG™ E (from Novozymes A/S); OPTIDEX™ 300 (from Genencor Int.); AMIGASE™ and AMIGASE™ PLUS (from DSM); G-ZYME™ G900, G-ZYME™ and G990 ZR (from Genencor Int.).

Glucoamylases may in an embodiment be added in an amount of 0.0001-20 AGU/g DS, preferably 0.001-10 AGU/g DS, especially between 0.01-5 AGU/g DS, such as 0.1-2 AGU/g DS.

### Biological Treatment

Microorganisms for additional biological treatment or pre-treatment may be selected among bacteria, yeasts or fungi, or mixtures thereof. The microorganisms or mixtures of two or more microorganisms may provide for an improved methane production in the anaerobic fermentation step of the biogas production process. Preferred examples of microorganisms according to the invention includes strains of the genus: Bacillus, Pseudomonas, Enterobacter, Rhodococcus, Acinetobacter, and Aspergillus such as Bacillus licheniformis, Pseudomonas putida, Enterobacter dissolvens, Pseudomonas fluorescens, Rhodococcus pyridinivorans, Acinetobacter baumanii, Bacillus amyloliquefaciens, Bacillus pumilus, Pseudomonas plecoglossicida, Pseudomonas pseudoacaligenes, Pseudomonas antarctica, Pseudomonas monteilii, Pseudomonas mendocina, Bacillus subtilis, Aspergillus niger and Aspergillus oryzae and any combinations or two or more thereof.

Particular preferred strains include: Bacillus subtilis (NRRL B-50136), Pseudomonas monteilii (NRRL B-50256), Enterobacter dissolvens (NRRL B-50257), Pseudomonas monteilii (NRRL B-50258), Pseudomonas plecoglossicida (ATCC 31483), Pseudomonas putida (NRRL B-50247), Pseudomonas plecoglossicida (NRRL B-50248), Rhodococcus pyridinivorans (NRRL 50249), Pseudomonas putida (ATCC 49451), Pseudomonas mendocina (ATCC 53757), Acinetobacter baumanii (NRRL B-50254), Bacillus pumilus (NRRL B-50255), Bacillus licheniformis (NRRL B-50141), Bacillus amyloliquefaciens (NRRL B-50151), Bacillus amyloliquefaciens (NRRL B-50019), Pseudomonas mendocina (ATCC 53757), Pseudomonas monteilii (NRRL B-50250), Pseudomonas monteilii (NRRL B-50251), Pseudomonas monteilii (NRRL B-50252), Pseudomonas monteilii (NRRL B-50253), Pseudomonas antarctica (NRRL B-50259), Bacillus amyloliquefaciens (ATCC 55405), Aspergillus niger (NRRL 50245), and Aspergillus oryzae (NRRL 50246).

The skilled person will appreciate how to determine suitable amounts of these preferred strains in uses according to the invention, using well known techniques. In preferred embodiments the strains are added in amounts in the range of 1.0×10⁶ to 5.0×10⁹ CFU/g.

As examples of particular preferred microorganisms or mixtures of two or more microorganisms can be mentioned:
A mixture containing: Bacillus subtilis (NRRL B-50136; 1.1×10⁹ CFU/g), Pseudomonas monteilii (NRRL B-50256; 0.6×10⁹ CFU/g), Enterobacter dissolvens (NRRL B-50257; 0.6×10⁹ CFU/g), Pseudomonas monteilii (NRRL B-50258; 0.8×10⁹ CFU/g), Pseudomonas fluorescens (ATCC 31483; 0.8×10⁹ CFU/g), Pseudomonas putida (NRRL B-50247; 0.4×10⁹ CFU/g), Pseudomonas plecoglossicida (NRRL B-50248; 0.4×10⁹ CFU/g), Rhodococcus pyridinivorans (NRRL 50249; 0.8×10⁹ CFU/g), Pseudomonas putida (ATCC 49451, 0.4×10⁹ CFU/g), Pseudomonas mendocina (ATCC 53757; 0.8×10⁹ CFU/g), and Acinetobacter baumanii (NRRL B-50254; 0.2×10⁹ CFU/g;
A mixture containing: Bacillus subtilis (NRRL B-50136; 1.6×10⁹ CFU/g), Bacillus pumilus (NRRL B-50255; 0.2×10⁹ CFU/g), Bacillus amyloliquefaciens (NRRL B-50141; 0.2×10⁹ CFU/g), Bacillus amyloliquefaciens (NRRL B-50151; 0.2×10⁹ CFU/g), Bacillus amyloliquefaciens (NRRL B-50019; 0.2×10⁹ CFU/g), Pseudomonas monteilii (NRRL B-50256; 0.2×10⁹ CFU/g), Enterobacter dissolvens (NRRL B-50257; 0.3×10⁹ CFU/g), Pseudomonas monteilii (NRRL B-50258; 0.8×10⁹ CFU/g), Pseudomonas plecoglossicida (ATCC 31483; 0.7×10⁹ CFU/g), Pseudomonas putida (NRRL B-50247; 0.2×10⁹ CFU/g), Pseudomonas plecoglossicida (NRRL B-50248; 0.2×10⁹ CFU/g), Rhodococcus pyridinivorans (NRRL 50249; 0.3×10⁹ CFU/g), Pseudomonas putida (ATCC 49451; 0.2×10⁹), Pseudomonas mendocina (ATCC 53757; 0.3×10⁹ CFU/g), Pseudomonas monteilii (NRRL B-50250; 0.1×10⁹ CFU/g), Pseudomonas monteilii (NRRL B-50251; 0.1×10⁹ CFU/g), Pseudomonas monteilii (NRRL B-50252; 0.1×10⁹ CFU/g), Pseudomonas monteilii (NRRL B-50253; 0.1×10⁹ CFU/g), and Pseudomonas antarctica (NRRL B-50259; 0.2×10⁹CFU/g); and
A mixture containing: Bacillus subtilis (NRRL B-50136; 3.5×10⁹ CFU/g), Bacillus amyloliquefaciens (ATCC 55405; 1.0×10⁹ CFU/g), Pseudomonas antarctica (NRRL B-50259; 0.2×10⁹ CFU/g), Aspergillus niger (NRRL 50245; 0.8×10⁹ CFU/g), and Aspergillus oryzae (NRRL 50246; 0.8×10⁹ CFU/g).

Further, the microorganism or mixture of two or more microorganisms commercially available from Novozymes Biological Inc. under the trade names: BI-CHEM ABR-Hydrocarbon, BI-CHEM DC 1008 CB and Manure Degrader are also suitable.

Incubation under aerobic conditions may be performed as batch process, fed batch process or continuous process. In a batch process the container is filled, a suitable inoculum of the microorganisms is added and the process proceeding for a desired time. In a fed batch process a initial volume of bagasse-derived material is added into the container, typically 25-75% of the total operational volume of the container, a suitable inoculum of the microorganism is added and the process is proceeding until a certain conversion/cell density is reached where additional feed in form of bagasse-derived material is added at a suitable rate and the process is continued until the container is full and optionally for an additional time without additional feed. In a continuous process the process is started by adding the material into the container and a suitable inoculum of the microorganism is added, when a desired cell density is reached a stream of the composition in the container is removed and simultaneously a stream of the material is added to the container so that the volume remains essentially constant and the process is continued in principle as long as desired. It may even be possible to use a combination of these techniques. These techniques are known within the art and the skilled person will appreciate how to find suitable parameters for a particular process depending on the particular dimensions and properties of the container.

Means for aeration are well known in the art and it is within the capabilities of the skilled person to select suitable means for aeration for the present invention. Usually aeration is performed by blowing atmospheric air through the composition typically via one or more tube(s) or pipe(s) located in the lower part of the container said one or more tube(s) or pipe(s) is/are provided with holes at regular intervals to provide for an even distribution of the air in the composition. Other means for aerating may also be used according to the invention.

The rate of aeration during the aerobic fermentation step is selected to provide for a convenient growth rate of the microorganisms. Rate of aeration may be measured in volume air per volume ferment per minute (v/v/m) and usually aeration in the range of 0.01 v/v/m to 10 v/v/m is suitable, preferably 0.05 v/v/m to 5 v/v/m, more preferred 0.1 v/v/m to 2 v/v/m, more preferred 0.15 v/v/m to 1.5 v/v/m and most preferred 0.2 v/v/m to 1 v/v/m.

The duration of this step will be decided taking into account that on one side the incubation under aerobic conditions should be continued for a sufficient long time to make a satisfactory part of the lignocellulosic soluble and available for the following microbial or biological process, on the other side the aerobic step should not be extended so long that a too large fraction of the fibre fraction is combusted. Usually the aerobic fermentation is continued for 5 to 30 days, preferably from 7 to 25 days, more preferred from 10 to 20 days and most preferred around 15 days. It has been found that using such an incubation period a suitable high fraction of the lignocellulosic fibres is converted into a form that can be converted in a following microbial or biological process.

The temperature in this step should be selected taking into account the particular requirements of the microorganism or mixture of two or more microorganisms used according to the invention. Usually the temperature is selected in the range of 10° C. to 60° C., preferably in the range of 15° C. to 50° C., more preferred in the range of 20° C. to 45° C., even more preferred in the range of 25° C. to 40° C. and most preferred about 35° C.

The method according to the invention increases the degradability of the bagasse-derived material making it more accessible for a following microbial or biological process such as for example a biogas production process leading to a higher yield than would have been possible without the method of the invention.

The incubation under aerobic conditions is continued until the degradability of the lignocellulosic fibres has been increased in a satisfactory extent so that a considerable high fraction of lignocellulosic fibres has been made accessible for a following microbial or biological process.

When lignocellulosic fibres have been made accessible according to the present invention the accessible fibres or part thereof will be available for the following microbial or biological process, meaning that the accessible fibres or part thereof can be converted in the following microbial or biological process. Thus, it can be determined if the accessability of lignocellulosic fibres have been increased by the method of the invention by performing a following microbial or biological process on the material treated according to the invention and comparing the yield of said following microbial or biological process with a corresponding following microbial or biological process using same material comprising lignocellulosic fibres but without the method of the invention.

A material comprising lignocellulosic fibres can be treated using a method of the invention, followed by a usual anaerobic biogas forming process and the yield of the biogas using the material comprising lignocellulosic fibres treated according to the invention can be determined and compared with the same biogas forming process but without the method of the invention. If the yield of biogas is higher using the method of the invention, according to the invention, the accessibility of the lignocellulosic fibres has increased. The skilled person will appreciate that the increased accessibility according to the invention can be determined in other ways using different following microbial or biological methods.

In one embodiment a method of the invention relates to the production of methane. In this embodiment the production of methane may be conducted as a two step process comprising a microbiological aerobic step and/or an enzymatic pre-treatment followed by a process for biogas production. In another embodiment the production of methane may be conducted as a two step process comprising a microbiological aerobic step and/or a pre-treatment followed by a process for biogas production with a simultaneous enzymatic treatment before or during the biogas production process. In principle, any process for biogas formation as known within the art may be used herein.

In another embodiment, the production of methane may be conducted as a process comprising a first process for biogas formation, followed by a microbiological aerobic step and/or an enzymatic treatment, again followed by a second process for biogas formation.

### EXAMPLES

### Example 1

### Substrate Characterization of Raw and Steam-Exploded Bagasse

Tests were performed on sugarcane bagasse which is a difficult biodegradable lignocellulosic rich substrate. The bagasse sample originated from a Brazilian sugarmill and was treated in an industrial scale steam explosion process (STEX).

Two different samples bagasse were obtained and charecterized, one was raw bagasse as such and the other steam explosion treated bagasse. The latter is raw bagasse that was submitted to high pressure and high temperature, with saturated steam at 170° C. in a closed reactor for 15 minutes, follow by quick relase of pressure at the end of treatment. This process is referred to as steam explosion is applied to increase the digestibility of bagasse, which is used as cattle fodder. For the analysis of the substrates, it was important to have a homogeneous sample of mm-size. Therefore, the samples of bagasse were shredded to further reduce particle size. This was done by means of a kitchen blender.

TABLE 1
Characterization of the raw bagasse sample and
of the bagasse sample after steam explosion.

| | | | Bagasse after |
|---|---|---|---|
| Parameter | Unit | Raw bagasse | steam explosion |
| Total Solids (TS) | % | 52.20-52.47 | 44.58-44.92 |
| Ash | % | 1.70-1.67 | 0.82-0.81 |
| Volatile solids (VS) | % | 50.50-50.80 | 43.76-44.11 |
| COD total | mg/g wet | 690-604 | 547-577 |
| | weight | | |
| pH | — | 6.4 | 5.0 |
| Volatile Fatty Acids | mg/L | 0 | 0 |
| VS/TS | % | 96.8 | 98.2 |

| | | |
|---|---|---|
| COD/TS | 1.24 | 1.26 |
| COD/VS | 1.28 | 1.28 |

Standard analyses were performed described as described in the art. In short, total solids (TS) was determined by drying at 105 degree Celsius until no further weight change occurred. Ash was determined in a muffle furnace by heating the sample to 600 degree Celsius in a crucible until no further weight change occurred. Volatile solids (VS) was calculated by subtracting total solids with ash content. Total volatile fatty acids was determined by the distillation and subsequent pH titration according to standard methods in the art (Standard Methods for the Examination of Water and Wastewater, Eaton et al., Amer Public Health Assn; 21, Oct. 15, 2005).

Chemical oxygen demand (COD) was measured by the potassium dichromate method as described by Greenberg et al. in Standard Methods for the examination of water and wastewater. 18th Edition, 1992, p. 5-7. The pH was measured by adding 1 g wet weight material to 100 ml distilled water and stirring for 24 hours, followed by pH measurement by calibrated pH electrode.

### Example 2

### Characterization of the Seeding Sludge

All the fermentation tests were started with the same type of thermophilic seeding sludge. For each test series, a fresh sample was collected. The latter originated from different thermophilic full-scale biogas installation plants, treating manure, slaughterhouse waste and/or by-products from food-processing industries.

At the start of the experiments, this seeding sludge was characterized in terms of biomass concentration as total suspended solids (TSS) and volatile suspended solids (VSS), residual soluble compounds (soluble COD and VFA) and specific methanogenic activity (SMA).

Samples of the seeding sludge were centrifuged for 10 min at 1750 g. The supernatant was discarded and the pellet was washed with deionized water to remove soluble salts. Samples were centrifuged again for 10 min at 1750 g. The resulting pellet was transferred to a crucible and dried to constant weight at 105° C. The samples were cooled in an exsicator and the TSS was determined by mass. Following TSS determination, the crucible was transfered to a muffeloven and heated to 600° C. for 2 hours. Following cooling in an exsicator, the mass of residual ash was determined. The VVS was determined by subtracting the residual ash from the TSS. In Table 2, an overview is presented of the main characteristics of a typical seeding sludge.

The specific methanogenic activity was measured according to a standard procedure: In an Erlenmeyer flask of 1.0 L, 800 ml of the sludge was brought together with a synthetic, easily biodegradable feed, consisting of 1.5 ml ethanol and 4 g sodium acetate, which cooresponds to approximatily 5 g of COD. The reactor was connected to a biogas column and placed in a hot water bath at 53° C. The volume and rate of biogas production were followed over time. At the end of the sludge activity test, the methane concentration of the biogas produced was analyzed and the specific methanogenic activity (SMA) was expressed in gram CH₄ calculated as COD per g VSS per day (COD/g VSS.d) based on the theoretical assumption that 1 g COD is converted into 0.35 L CH₄. The results of a sludge activity test of the inoculum are summarized in Table 2.

TABLE 2
Overview of the main characteristics of the seeding sludge.

| | Unit | Value |
|---|---|---|
| Total Suspended Solids TSS | g/L | 54.48 |
| Volatile Suspended Solids VSS | g/L | 21.48 |
| VSS/TSS | % | 39 |
| Total Solids TS | g/L | 78.26 |
| Ash | g/L | 29.01 |
| Ash/TS | % | 37 |
| COD soluble | mg/L | 22427 |
| Volatile Fatty Acids | mg/L | 343 |
| Specific Methanogenic activity test: | | |
| Biomass (in VSS) | g | 17.2 |
| Biogas after 1.1 days | L | 3.7 |
| CH₄ | % | 81.7 |
| CH₄ after 1.1 days | L | 3.0 |
| SMA (interval start −>20 h) | g CH₄•COD/g VSS.d | 0.61 |

For thermophilic seeding sludge, the VSS concentration was considered as the best approach to measure the active anaerobic biomass concentration. Yet, it should be noted that for this measurement, there is interference of residual non-degraded organic particular matter from the substrate, which was previously fed to this biomass. Moreover, centrifugation was required to separate the biomass from the liquid phase.

The results of the sludge activity test indicated that the thermophilic seeding sludge, used for the anaerobic experiments, had a satisfying specific methanogenic activity. The thermophilic sludge was highly pH buffered.

### Example 3

### Batch Anaerobic Digestion of Steam-Exploded Bagasse and Raw Bagasse

To evaluate the effect of the steam explosion treatment of the bagasse in a biogas process, the following experiment was carried out:

500 ml bottles were inoculated with 200 ml of thermophilic anaerobic sludge obtained from a full scale biogas plant running on manure and industrial waste. Substrate, either in the form of raw or steam-exploded bagasse, was added to the bottles which were then flushed with nitrogen and sealed with butyl rubber stoppers to ensure anaerobic conditions and an airtight seal.

The bottles were incubated at 53° C. until no further methane production was observed. During the course of the experiment the bottles were vented on a regular basis to avoid the build-up of pressure in the bottles. Before venting, the precise amount of methane in each flask was determined to allow for accurate quantification of the methane produced throughout the experiment.

To analyze the methane formation, samples of the bottle headspace volumes were taken with a Hamilltion gas syringe and subjected to GC analysis on a Varian 3900 gas chromatograph with a PoraPLOT Q (10 μm) 25 m×0.32 mm fused silica separation column (Varian, Agilent Technologies, USA). Sample vials used for all gas samples were Supelco, Precleaned 2 cm³ Clear Screw Cap Vials (Supelco, Bellafonte, Pa., USA). Gas samples were quantified by comparison to a standard curve obtained with methane gas standards (Mikrolab, Aarhus, Denmark).

Three different anaerobic digestions were performed:
1) Anaerobic sludge only
2) Anaerobic sludge with 1.67 g (dry weight) raw bagasse
3) Anaerobic sludge with 1.67 g (dry weight) steam-exploded bagassse

Three independent biological replicas were made for each treatment and two replicas were done for all methane measurements and standards. The final results of the three digestions are shown in table 3.

TABLE 3
Final methane yields obtain in batch experiment on
bagasse. Methane data given at standard condition
(20° C., 1 atm.). The background methane production
from the inoculation sludge has been subtracted.

| | Methane | | | |
|---|---|---|---|---|
| | produced | Total | | |
| | after | substrate | Yield | |
| Substrate | 14 days (ml) | dry mass (g) | ml CH₄/g TS | Increase |
| Raw bagasse | 264 | 1.67 | 158 | |
| Steam-exploded | 318 | 1.67 | 190 | 20.5% |
| bagasse | | | | |

Based on the data obtained it was clear that steam explosion of bagasse leads to a significant increase in the anaerobic digestibility, both with respect to initial conversion rate and the maximum obtainable conversion. The effect observed in this example after subtraction of the sludge baseline methane production was a 20.5% increase in methane yield from steam-exploded bagasse compared to raw bagasse.

### Example 4

### Batch Digestion of Steam-Exploded Bagasse with Enzymatic Pretreatment

In a second series of anaerobic digestion tests, the steam-exploded bagasse samples were used as substrate. The intention was to investigate if the physical pre-treatment by steam explosion disrupted the cell wall structure of the bagasse to such an extent that it made the lignocellulosic material more accessible to enzymes. The aim of this test series was to examine the effect of enzyme addition dosed either before fermentation or directly in the fermentation reactor on the digestion of steam-exploded bagasse.

In order to assess the effect of the addition of enzymes on the anaerobic biodegradability of lignocellulosic material, several series of thermophilic (53° C.) short-term batch-tests were performed on lab-scale.

Each reactor set-up consisted of a 1.0 L Erlenmeyer flask placed in a thermostatic hot waterbath (temperature regulated at 53° C.) and connected to a biogas column for the collection and measurement of biogas produced.

At the start of each test series, the reactors were seeded with the same amount of fresh thermophilic anaerobic sludge. The feeding of the seeding sludge with a certain amount of different lignocellulosic substrates was done manually batchwise. After the feeding and pH-measurement of the mixed liquors, each reactor was connected to a column to follow the biogas production. At the end of the digestion period (about 1 week or more per feeding cycle), samples were taken to analyze the methane concentration of the produced biogas by gas chromatography. For each treatment, three to four successive feedings with the same substrate were performed.

Enzymatic treatment was carried out either as an enzymatic pretreatment prior to anaerobic digestion or by direct enzyme addition to the reactor. In both cases, the enzymes were a mixture of a two enzyme products A and B.

Enzyme A is a a Trichoderma reesei cellulase preparation containing Aspergillus oryzae beta-glucosidase fusion protein (WO 2008/057637) and Thermoascus aurantiacus GH61A polypeptide (WO 2005/074656). Enzyme B is an Aspergillus aculeatus GH10 xylanase (WO 94/021785).

Enzymatic pretreatment was done at 5% TS of the steam-exploded bagasse (or 110 g wet weight pre-treated bagasse+1 L tap-water) in a closed vessel. Each vessel was placed on a magnetic stirrer and stirred continuously throughout the enzymatic hydrolysis treatment. The total enzymatic hydrolysis time was set to 48 hours with the temperature regulated to 50° C. during the whole period. Enzymes were dosed at: Enzyme A=59 mg product/g TS (or 8.9 mg enzyme protein/g TS) and Enzyme B=2.5 mg product/g TS (or 0.1 mg enzyme protein/g TS).

The following tests were carried out:
Background test: Seeding sludge without addition of substrate
Control test: Seeding sludge+steam-exploded bagasse (influent at 5% TS)
Test 1: Seeding sludge+enzymatically pre-treated steam-exploded bagasse
Test 2: Seeding sludge+steam-exploded bagasse+enzyme mix.

For all the tests with the bagasse substrate, including the control test, bagasse in tap-water at a concentration of about 5% TS (110 g wet weight pre-treated bagasse+1 L tap-water) was applied as feeding. At the start, the 4 reactors were seeded with the same amount of thermophilic sludge, namely 800 ml, corresponding with a biomass concentration of about 17 g VSS. Steam-exploded bagasse was added as substrate. The input of bagasse in the control reactor and in the two test reactors amounted to about 5 g of total solids per feeding cycle. The corresponding amount of total COD input ranged around 6.2 g COD per feeding cycle.

Four successive feedings (feeding cycles) with the same amount of bagasse input were tested. After each feeding, a digestion period of 7 to 8 days was taken into account. At the end of each feeding cycle, the methane concentration of the biogas produced and the residual soluble COD and VFA concentrations were measured. The total cumulative results for the 4 feeding cycles are summarized in Table 4.

TABLE 4
Summary of the digestion tests with steam-exploded bagasse as
substrate; the upper part summarizes results for steam-exploded
bagasse at 5 g TS and 6.2 g COD; the lower part summarizes results
for steam-exploded bagasse at 20 g TS and 24.8 g COD

| General | | | | | | |
|---|---|---|---|---|---|---|
| parameters | Control | | Test 1 | | Test 2 | |
| Enzyme addition | No | | | | | |
| Type enzyme | — | | A/B | | A/B | |
| Amount enzyme* | — | | 255 μL/11 μL | | 255 μL/11 μL | |
| Extra COD enzyme | — | | 94 | mg | 94 | mg |
| Time of enzyme | — | | Before | | During | |
| addition | | | fermentation | | fermentation | |
| Enzyme addition | No | | | | | |
| Type enzyme | — | | A/B | | A/B | |
| Amount enzyme | — | | 1.02 mL/44 μL | | 1.02 mL/44 μL | |
| Extra COD enzyme | — | | 376 | mg | 376 | mg |
| Time of enzyme | — | | Before | | During | |
| addition | | | fermentation | | fermentation | |
| Digestion period | 27 | days | 27 | days | 27 | days |
| Total Biogas | 7.43 | L | 9.77 | L | 10.31 | L |
| volume* | | | | | | |
| ‘Net’ Biogas | 7.43 | L | 9.58 | L | 10.12 | L |
| volume** | | | | | | |
| Methane | 68.1% | | 67.5% | | 67.7% | |
| concentration | | | | | | |
| ‘Net’ methane | 4.98 | L | 6.47 | L | 6.78 | L |
| volume | | | | | | |

| | | |
|---|---|---|
| INCREASE | 30% | 36% |

*Biogas volume after subtracting the biogas volume of the background test at 32 degree Celcius, 1 atm.
**Net biogas volume taking into account the total conversion of the enzyme COD.

Based on the data give in table 4 it is evident that the addition of the enzyme mix, dosed either during the digestion or in a previous seperate pre-treatement, considerably improved the total biogas- and methane productions by 30% (Test 1) to 36% (Test 2). Any effect based on the difference between the time of dosage of these enzymes (in a separate pre-treatment or directly in the anaerobic reactor) was less pronounced in this experiment. However, based on lab-scale tests with pre-treated bagasse as substrate, an important positive net effect of the enzymes was found.

### Example 5

### Long Term Semi Continuous Anaerobic Digestion of Steam-Exploded Bagasse with Either Enzymatic Pretreatment or Direct Enzyme Addition

In order to assess the positive effect of enzyme addition on the continuous anaerobic thermophilic digestion of steam-exploded bagasse on a longer term and to experimentally determine the impact of enzyme addition on the volumetric loading rate, the removal efficiencies, the biogas- and methane production and the overall performance of the anaerobic reactor, treating steam-exploded bagasse, 3 (semi-) continuous reactors was started and operated for 4 months.

Each experimental set-up of these semi-continuous CSTR (completely stirred tank reactor) tests consisted of an anaerobic reactor with an active reactor volume of 1.8 L, placed in a thermostatic hot water bath at 52° C. and connected to a biogas column to follow the biogas production. The liquor of each reactor was continuously mixed by means of a stirring device to enhance the contact between the substrate and the biomass and to prevent settling and accumulation of solids in the reactors. Because of the nature of the steam-exploded bagasse substrate, the feeding was done manually, yet on a frequent basis three times a week. Prior to the feeding, the same amount of digested material (mixed liquor) was manually withdrawn from each reactor. The steam-exploded bagasse was dosed in the form of 8% TS. The sludge used to inoculate the 3 continuous reactors was well-adapted to the pretreated bagasse as feed substrate and was taken from a anaerobic digestor that had been running of bagasse for several months.

The enzyme mix used in this experiment was a blend of an Aspergillus aculeatus GH10 xylanase (WO 94/021785) and a Trichoderma reesei cellulase preparation containing Aspergillus fumigatus beta-glucosidase (WO 2005/047499) and Thermoascus aurantiacus GH61A polypeptide (WO 2005/074656). The dosage was 55 mg enzyme blend/g TS (or 7.8 mg enzyme protein/g TS).

To examine the effect of the enzyme on the anaerobic conversion of steam-exploded bagasse and to determine the effect of the dosage point of this enzyme (directly in the anaerobic reactor or during a specific pre-treatment), three similar CSTR reactors were operated simultaneously during a period of 111 days (after start-up week).

The first reactor acted as the control reactor and was fed only with the substrate as such, the second and third reactor were operated as the test reactors with the same enzyme addition. In both test reactors, the same enzyme dosage was applied during the whole test period. In the first test reactor, the enzyme was always added directly in the reactor after each batchwise feeding. The feedstream for the second test reactor was previously submitted to a pre-treatment: The bagasse was brought in with tap water (about 8% TS), the same dosage of enzyme was added, the pH was adjusted to about 5 and the influent vessel was placed on a magnetic stirrer with continuous mixing and in an incubator at 50° C. during a period of 2 days.

In order to be able to deduce the net effect of the enzyme addition on to the anaerobic digestion, the two other influents were also submitted to the same pre-treatment, except for the enzyme addition.

The tests with the pre-treatment and enzyme addition were carried out over a period of 4 months. During the first two months, the three reactors were operated under stable conditions at the same relativly low volumetric loading rate. The three CSTR reactors were started at a volumetric loading rate of about 2.3 g TS/L reactor per day, corresponding with a hydraulic and sludge retention time of 35 days.

In the following weeks, the volumetric loading rate of the reactors was stepwise increased by augmentation of the amount of STEX (steam exploded) bagasse and tap water. Since the STEX bagasse was always added under the form of 8% TS, the increase of bagasse feed was accompanied by a decrease of the hydraulic and sludge retention time in the reactors. There was never any difference in loading rate between the three reactors.

To follow reactor performance, the effluents of each reactor were gathered on a weekly basis. The main characteristics (TSS, VSS, COD soluble, TAN, conductivity and volatile fatty acids) were determined at the end of each test period.

The reactors were supplemented with nitrogen(NH₄Cl) and phosphorous (KH₂PO₄) because of the low N and P content of the steam-exploded bagasse substrate. In addition to the nitrogen and phosphorous suplements, a mixture of trace elements was also added to the reactors to ensure that no micronutrients would become limiting.

The total test period was divided into 5 sub-periods on the basis of the volumetric loading rate. In Table 5, a general overview of the process parameters and resulting biogas production of the three continuous reactors is shown for each sub-period in the experiment.

TABLE 5
Overview of feeding and biogas production of the 3 CSTR reactors during test period.

| | Test | Test | Test | Test | Test |
|---|---|---|---|---|---|
| Parameter | period 1 | period 2 | period 3 | period 4 | period 5 |
| Days | 8-63 | 64-82 | 83-96 | 97-110 | 111-119 |
| Reactor volume (l) | 1.80 | 1.80 | 1.80 | 1.80 | 1.80 |
| Substrate: STEX bagasse | | | | | |
| (average for period) | | | | | |
| Wet Weight (g/day) | 7.3-7.5 | 10.0 | 12.0 | 14.5 | 18.0 |
| Total Solids (g/day) | 4.0-4.2 | 5.6 | 6.7 | 8.1 | 10.0 |
| COD (g/day) | 5.2-5.4 | 7.2 | 8.6 | 10.4 | 12.9 |
| Influent Flow rate** | 28 | 38 | 46 | 56 | 69 |
| (ml/l reactor per day) | | | | | |
| Hydraulic Retention Time | 36 | 26 | 22 | 18 | 14 |
| (HRT; days) | | | | | |
| Volumetric Loading Rate | | | | | |
| (Bv) | | | | | |
| (g TS/l reactor per day) | 2.28-2.33 | 3.08 | 3.70 | 4.48 | 5.56 |
| (g COD/l reactor per day) | 2.87-3.01 | 3.98 | 4.78 | 5.78 | 7.17 |
| Biogas | | | | | |
| Control | | | | | |
| (l/l reactor per day) | 0.73 ± 0.08 | 0.86 ± 0.13 | 1.09 ± 0.06 | 1.20 ± 0.05 | 1.28 ± 0.07 |
| (l/g TS) | 0.32 ± 0.04 | 0.28 ± 0.04 | 0.30 ± 0.01 | 0.27 ± 0.01 | 0.23 ± 0.01 |
| (% methane) | 63.5 ± 3.6 | 66.6 ± 3.3 | 67.5 ± 3.7 | 65.9 | 72.9 |
| Reactor 1 (enz. addition) | | | | | |
| (l/l reactor per day) | 0.80 ± 0.09 | 0.99 ± 0.13 | 1.15 ± 0.05 | 1.41 ± 0.10 | 1.67 ± 0.04 |
| (l/g TS) | 0.35 ± 0.03 | 0.32 ± 0.04 | 0.31 ± 0.01 | 0.31 ± 0.02 | 0.30 ± 0.01 |
| (% methane) | 67.0 ± 4.6 | 71.2 ± 5.8 | 70.9 ± 1.2 | NA | 71.8 |
| Reactor 2 (enz. pretreat) | | | | | |
| (l/l reactor per day) | 0.77 ± 0.08 | 0.92 ± 0.11 | 1.15 ± 0.03 | 1.42 ± 0.05 | 1.38 ± 0.27 |
| (l/g TS) | 0.34 ± 0.03 | 0.20 ± 0.04 | 0.31 ± 0.01 | 0.32 ± 0.01 | 0.25 ± 0.05 |
| (% methane) | 64.4 ± 6.5 | 71.8 ± 0.9 | 73.9 ± 1.4 | 76.2 | 72.6 |

TABLE 6
Overview of effluent composition from the 3 CSTR reactors
duing test period; all measurements are in mg/l.

| | Test | Test | Test | Test | Test |
|---|---|---|---|---|---|
| Parameter | period 1 | period 2 | period 3 | period 4 | period 5 |
| Days | 8-63 | 64-82 | 83-96 | 97-110 | 111-119 |
| COD soluble | | | | | |
| Control | 8821 ± 1022 | 11510 ± 576 | 10510 ± 1253 | 11534 ± 2600 | 9186 |
| Reactor 1 | 6797 ± 313 | 8665 ± 222 | 8271 ± 552 | 8441 ± 974 | 8868 |
| Reactor 2 | 6993 ± 368 | 8011 ± 708 | 8880 ± 1356 | 6824 ± 248 | 7746 |
| VFA | | | | | |
| Control | 1110 ± 304 | 995 ± 162 | 891 ± 11 | 618 ± 152 | 635-753 |
| Reactor 1 | 193 ± 269 | 71 ± 105 | 28 ± 1 | 22 ± 8 | 0 |
| Reactor 2 | 463 ± 432 | 183 ± 138 | 192 ± 223 | 90 ± 74 | 754-1900 |

TABLE 7
Cumulative results of the feeding and biogas productions of the 3
continuous CSTR tests; total test period from day 8 until day 119.

| | | Test reactor | Test reactor |
|---|---|---|---|
| Parameter | Control | 1 | 2 |
| Addition enzyme | No | Directly | Pre-treatment |
| Time (days) | 111 | 111 | 111 |
| Total values | | | |
| Total feeding/reactor | | | |
| (g WW) | 1125 | 1125 | 1125 |
| (g TS) | 624 | 624 | 624 |
| (ml water) | 7772 | 7772 | 7772 |
| Total loading/reactor | 346.7 | 346.7 | 346.7 |
| Total biogas/reactor | | | |
| (liter in total) | 175.3 | 198.6 | 190.1 |
| (%) | 100 | 113 | 108 |
| Average values | | | |
| Volumetric loading rate Bv | | | |
| (g TS/l reactor per day) | 3.12 ± 1.07 | 3.12 ± 1.07 | 3.12 ± 1.07 |
| Biogas | | | |
| (l/l reactor per day) | 0.90 ± 0.23 | 1.02 ± 0.30 | 0.98 ± 0.28 |
| (l/g TS added) | 0.30 ± 0.04 | 0.33 ± 0.04 | 0.32 ± 0.04 |

TABLE 8
Cumulative results of the feeding and biogas productions
of the 3 continuous CSTR tests; test period of higher
loading rate from day 64 until day 119.

| | | Test | Test |
|---|---|---|---|
| Parameter | Control | reactor 1 | reactor 2 |
| Addition enzyme | No | Directly | Pre-treatment |
| Time (days) | 55 | 55 | 55 |
| Total values | | | |
| Total feeding/reactor | | | |
| (g WW) | 723 | 723 | 723 |
| (g TS) | 401 | 401 | 401 |
| (ml water) | 5000 | 5000 | 5000 |
| Total loading/reactor | 223.0 | 223.0 | 223.0 |
| Total biogas/reactor | | | |
| (liter in total) | 103.4 | 119.8 | 113.9 |
| (%) | 100 | 116 | 110 |
| Average values | | | |
| Volumetric loading rate Bv | | | |
| (g TS/l reactor per day) | 3.98 ± 0.88 | 3.98 ± 0.88 | 3.98 ± 0.88 |
| Biogas | | | |
| (l/l reactor per day) | 1.08 ± 0.18 | 1.26 ± 0.26 | 1.19 ± 0.24 |
| (l/g TS added) | 0.27 ± 0.03 | 0.31 ± 0.03 | 0.30 ± 0.04 |

The impact of the enzyme on the thermophilic digestion of the STEX bagasse in a CSTR reactor was investigated in a long-term lab-scale test. Three similar reactors (1.8 L of active reactor volume) were operated at gradually increasing loading rates, starting from 2.25 g TS/L reactor per day and stepwise augmented up to 5.56 g TS/I reactor per day. It concerned a control reactor with no enzyme addition, a test reactor with enzyme addition directly in the anaerobic reactor and a test reactor with enzyme addition in a specific pre-treatment (2 days stirring at 50° C. and pH of about 5). The performance of these reactors was followed by measurements of biogas and methane production on the one hand and characterization of the anaerobic effluents.

When the results of the total test period of 111 days were taken into account, an overall increase of the total volume of biogas production with 13% was obtained in Test reactor 1 with enzyme addition directly in the reactor. A lower effect but still significant effect on the total biogas production of 8% increase was obtained in the other test reactor with enzyme addition in the pre-treatment (Table 7).

A higher positive impact of the enzyme addition was achieved when the CSTR reactors were operated at higher volumetric loading rates. Therefore, the total and average biogas productions were also calculated for the test period from d 64 until d 119 with volumetric loading rates ranging between 3.08 and 5.56 g TS/L reactor.d. The increase in total biogas production compared to the control test amounted to 16% in Test reactor 1 and 10% in Test reactor 2. Furthermore, it should be noted that the methane yield (I/g TS) was kept at a constant level of 0.30 to 0.31 in Test reactor 1 during test period 3 to 5, while the methane yield in the control reactor was lowered from 0.3 in test period 3 to 0.23 in test period 5. Hence, the best and most stable positive effect of the enzyme was obtained when the enzymes was added directly in the anaerobic reactor (Table 8).

From the results of the different sub-periods, it could be derived that the higher the loading rate, the more pronounced the impact of the enzyme addition was. The mutual differences in biogas production between the test reactors and the control reactor in test periods of high loading rates, were mainly due to an incomplete conversion of the STEX bagasse in the control test (overloading). The latter resulted in gradually decreasing biogas productions per g of TS STEX bagasse added in the control test. Yet, in the test reactor with enzyme addition directly in the reactor, the amount of biogas per g of TS added remained more or less constant (about 0.3 L biogas/g TS added). The addition of the enzyme in the pre-treatment gave rise to more fluctuating results. Overall, the impact of the enzyme on the biogas production was lower in this test (i.e. with enzymes added during pretreatment) compared to when the enzymes was added directly, but still significantly improved compared to the control reactor.

The better performance of the test reactors with direct enzyme addition, especially at the higher loading rates tested, was confirmed by the lower effluent VFA and soluble COD concentrations. Also for these parameters, the lowest and most stable results were achieved in the test reactor with enzyme addition directly in the reactor (Table 6).

Overall, the main conclusion of this long-term lab-scale test was the enzyme, when added directly in the anaerobic reactor guaranteed a more stable and efficient performance of the thermophilic anaerobic CSTR reactor treating steam exploded bagasse (8% TS). Especially in periods of high loading rates, significantly higher biogas and methane productions, as well as lower residual soluble compounds, were achieved in the tests, indicating the positive impact of the enzyme addition on the anaerobic conversion of steam exploded bagasse.

1. A biogas production process comprising the steps of providing a slurry comprising a bagasse-derived material and water, and:
(a) performing a pre-treatment comprising adding one or more enzyme to the slurry to degrade the bagasse-derived material at a suitable temperature and pH, and then adding the enzyme-degraded material to a biogas digester tank at a suitable rate and ratio to effectively convert the material to biogas in the digester; or
(b) adding one or more enzyme to the slurry before adding the slurry to a biogas digester tank; or
(c) adding one or more enzyme to the digester tank after adding the slurry to the digester tank, to degrade the bagasse-derived material at a suitable temperature and pH and to effectively convert the material to biogas in the digester.

2. The process of claim 1, wherein the one or more enzyme is selected from the group consisting of an amylolytic enzyme, a lipolytic enzyme, a proteolytic enzyme, a cellulolytic enzyme, an oxidoreductase and a plant cell-wall degrading enzyme.

3. The process of claim 2, wherein the one or more enzyme is selected from the group consisting of aminopeptidase, alpha-amylase, amyloglucosidase, arabinofuranosidase, arabinoxylanase, beta-glucanase, carbohydrase, carboxypeptidase, catalase, cellobiohydrolase, cellulase, chitinase, cutinase, cyclodextrin glycosyltransferase, ferulic acid esterase, deoxyribonuclease, endo-cellulase, endo-glucanase, endo-xylanase, esterase, galactosidase, beta-galactosidase, glucoamylase, glucose oxidase, glucosidase, haloperoxidase, hemicellulase, invertase, isomerase, laccase, ligase, lipase, lyase, mannanase, mannosidase, oxidase, pectate lyase, pectin lyase, pectin trans-eliminase, pectin ethylesterase, pectin methylesterase, pectinolytic enzyme, peroxidase, protease, phytase, phenoloxidase, polygalacturonase, polyphenoloxidase, proteolytic enzyme, rhamnogalacturonan lyase, rhamnoglucanase, rhamnogalacturonase, ribonuclease, SPS-ase, transferase, transglutaminase, xylanase and xyloglucanase.

4. The process of claim 2, wherein the one or more enzyme is a protease, a pectate lyase, a ferulic acid esterase and/or a mannanase.

5. The process of claim 1, wherein the one or more enzymes is selected from a Trichoderma reseei cellulase preparation containing Aspergillus oryzae beta-glucosidase fusion protein and Thermoascus aurantiacus GH61A polypeptide and/or an Aspergillus aculeatus GH10 xylanase.

6. The process of claim 1, wherein the bagasse-derived material or the slurry is homogenized prior to or during step (a) or prior to step (b).

7. The process of claim 6, wherein a base is added to the bagasse-derived material or the slurry prior to or while it is being homogenized.

8. The process of claim 1, wherein the content of bagasse-derived material in the slurry is adjusted by continuous or stepwise addition of material to the slurry during step (a) or (b).

9. The process of claim 1, wherein the lignocellulose-containing material constitutes above 2.5% wt-% DS of the slurry.

10. The process of claim 1, wherein the bagasse-derived material is degraded at a pH in the range from 7 to 10.

11. The process of claim 1, wherein the bagasse-derived material is degraded at a temperature in the range from 20°-70° C.

12. The process of claim 1, wherein a solids separation step is performed in step (a) after the bagasse-derived material is degraded but before it is added to the digester tank, to purge not-solubilized solids and optionally feed them back into step (a) of the process.

13. The process of claim 1, wherein the bagasse-derived material prior to step (a) or (b) has been subjected to a microwave and/or an ultrasonic irradiation treatment.

14. The process of claim 1, wherein the bagasse-derived material has been chemically, mechanical and/or biologically treated prior to step (a) or (b).
